(19) United States
(12) Patent Application Publication
(10) Pub. No.: US 2026/0260431 A1
(43) Pub. Date: Sep. 3, 2026

(54) SURGICAL ANALYTICS AND TOOLS

(71) Applicant: Kaliber Labs Inc., San Francisco, CA (US)

(72) Inventors: Mason STALLMO, San Francisco, CA (US); James WACLAWIK, San Francisco, CA (US); Mark RUIZ, San Francisco, CA (US); Chandra JONELAGADDA, San Francisco, CA (US)

(21) Appl. No.: 18/878,669

(22) PCT Filed: Jun. 26, 2023
(86) PCT No.: PCT/US2023/069060

§ 371 Date: Dec. 24, 2024

Related U.S. Application Data
(60) Provisional application No. 63/355,563, filed on Jun. 24, 2022.

(51) Int. Cl. G06T 19/00; A61B 1/00; A61B 34/10; A61B 90/00; G06V 10/82; G06V 20/40; G16H 15/00

(52) U.S. Cl. CPC G06T 19/003; A61B 34/10; A61B 90/361; G06V 10/82; G06V 20/44; G06V 20/47; G16H 15/00; A61B 1/00016; A61B 2034/107; A61B 2090/376; G06T 2210/41

(57) ABSTRACT

Methods, apparatuses, and systems are disclosed for managing and processing surgical media, including surgical video data. In some variations, one or more neural networks may be trained to determine, locate, and/or identify surgical Receive patient data, and surgery events, tools, or procedures within the surgical video data.

### CLAIM OF PRIORITY

This patent application claims priority to U.S. provisional patent application No. 63/355,563, entitled “SURGICAL ANALYTICS AND TOOLS”, filed on Jun. 24, 2022, herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Many surgical procedures, such as arthroscopic joint surgical procedures, involve a camera that provides real-time surgical video data. The surgical video data may be shown on a display and be used to guide actions of the surgeon, doctor, or other clinician performing the surgery. In some cases, the surgical video data may be recorded and reviewed after the surgical procedure has been performed to review the outcome of the procedure.

The recorded surgical video data may include a vast amount of information, but analysis of the information may be time consuming. Furthermore, post-surgery analytics may not provide timely information to the surgeon during the time of the surgery.

### SUMMARY OF THE DISCLOSURE

The present disclosure relates generally to surgery and more specifically to managing and analyzing surgical media.

Described herein are apparatuses (including systems and devices, which may further include software, hardware and/or firmware), and methods to manage and process surgical analytic data, particularly surgical data associated with anatomical joint surgery. Surgical analytic data, such as video data from an endoscopic or arthroscopic camera, may be processed according to one or more trained neural networks. The trained neural networks may determine one or more surgical aspects from the surgical video data. In some examples, the surgical video data may be real-time video data enabling the trained neural networks to provide real-time analysis and surgical assistance.

Any of the methods described herein may manage surgical media. Any of the methods described herein may include receiving surgical video data, determining, via a processor executing a first neural network, one or more surgical events based on the surgical video data; and generating, via the processor executing a second neural network, a report summary based on the one or more determined surgical events.

In any of the methods described herein, the report summary may include video clip highlights of a surgical procedure. In any of the methods described herein, the first neural network may be trained to recognize at least one of a surgery stage, a surgical activity, and a surgical action. In some variations, in any of the methods described herein the first neural network may be trained to recognize at least on or a surgical tool, an implant, a suture, and an implant structure. In any of the methods described herein, the second neural network may be trained to indicate that no surgical progress was made (including made during the same or a related surgery) in the report summary.

Any of the methods described herein may further include receiving patient-specific radiological imaging information and generating a three-dimensional (3D) navigational guidance for a surgery based, at least in part, on the patient-specific radiological imaging information.

Furthermore, the 3D navigational guidance may be based at least in part on a patient-specific preoperative plan. In some variations, the 3D navigational guidance may be based at least in part on the surgical video data.

Any of the methods described herein may include generating a report summary that includes a text summary of one or more surgical stages within the surgical video data. Any of the methods described herein may include generating a log of pinless landmarks used and measurements taken during a surgery associated with the surgical video data.

Any of the methods described herein may include training the first neural network or the second neural network with the surgical video data. In any of the methods described herein, the report summary may be saved within a secure cloud-based storage unit.

In any of the methods described herein the surgical video data may be a real-time video stream received from an endoscopic camera. In some variations, the surgical video data may be received from a secure cloud-based storage unit. In some other variations, the surgical video data may be received via an HDMI or USB connector.

Any of the methods described herein may also include generating an alert based on a recognized surgical activity. Any of the methods described herein may also include receiving a voice input from a surgeon during a surgery and generating context specific alerts based on the voice input. In any of the methods described herein, the first neural network may be trained to determine a start and a stop of a surgical action. For example, the method or apparatuses described herein may be configured to identify the general/generic beginning of the surgery (surgical activity) and to progressively and/or separately identify more specific surgical procedures. In any of these methods and apparatuses the technique may parse or divide the identified surgical portions into (linked) stages, and may use this for surgical activity detection and/or analysis.

Any of the systems described herein may include one or more processors and a memory configured to store instructions that, when executed by one of the one or more processors, cause the system to receive surgical video data, determine, via a processor executing a first neural network, one or more surgical events based on the surgical video data, and generate, via a processor executing a second neural network, a report summary based on the one or more determined surgical events.

Any of the non-transitory computer-readable storage mediums described herein may include instructions that, when executed by one or more processors of a device, cause the device to perform operations including receiving surgical video data, determining via a processor executing a first neural network, one or more surgical events based on the surgical video data, and generating, via a processor executing a second neural network, a report summary based on the one or more determined surgical events.

For example, described herein are methods for managing surgical data that include: receiving surgical video data; applying a trained first neural network to the surgical video data to determine one or more surgical events based on the surgical video data, wherein the is first trained neural network is trained to recognize at least one of: a surgery stage, a surgical activity, a surgical action, a surgical tool, an implant, a suture, and an implant anchor; and forming a report summary including a video clip comprising highlights of one or more determined surgical using a second trained neural network wherein the second trained neural network is trained to identify a representative image or video clip from the surgical video data based on the one or more determined surgical events; and outputting the report summary. The output may be any appropriate output, including displaying, transmitting and/or storing.

The second neural network may be trained to indicate that no surgical progress was made during a related surgery in the video clip.

Any of these methods may include receiving patient-specific radiological imaging information and generating a three-dimensional (3D) navigational guidance for a surgery based, at least in part, on the patient-specific radiological imaging information. For example, the 3D navigational guidance may be based at least in part on a patient-specific preoperative plan. The 3D navigational guidance may be based at least in part on generic models of an anatomical joint.

Any of these methods may include generating a deidentified video clip based at least in part on the surgical video data. The report summary may include a text summary of one or more surgical stages within the surgical video data.

In any of these methods, forming the report summary may comprise generating a log of pinless landmarks used and measurements taken during a surgery associated with the surgical video data.

Outputting may comprise saving the report summary to a secure cloud-based storage unit. The surgical video data is a real-time video stream received from an endoscopic camera. The surgical video data may be received from a secure cloud-based storage unit. In some examples the surgical video data is received via an HDMI or USB connector.

Any of these methods may include generating an alert based on a recognized surgical activity. In some examples, the method may include: receiving a voice input from a surgeon during the surgery; and generating context specific alerts based on the voice input. The first neural network may be trained to determine a start and stop of a surgical action.

Also described herein are systems configured to perform any of these methods. For example, a system may include: one or more processors; and a memory configured to store instructions that, when executed by one of the one or more processors, cause the system to: apply a trained first neural network to a surgical video data to determine one or more surgical events based on the surgical video data, wherein the is first trained neural network is trained to recognize at least one of: a surgery stage, a surgical activity, a surgical action, a surgical tool, an implant, a suture, and an implant anchor; and form a report summary including a video clip comprising highlights of one or more determined surgical using a second trained neural network wherein the second trained neural network is trained to identify a representative image or video clip from the surgical video data based on the one or more determined surgical events; and output the report summary.

Also described herein are software, firmware, and/or hardware configured to perform any of these methods. For example, described herein are non-transitory computer-readable storage media comprising instructions that, when executed by one or more processors of a device, cause the one or more processors to perform operations comprising: applying a trained first neural network to a surgical video data to determine one or more surgical events based on the surgical video data, wherein the is first trained neural network is trained to recognize at least one of: a surgery stage, a surgical activity, a surgical action, a surgical tool, an implant, a suture, and an implant anchor; and forming a report summary including a video clip comprising highlights of one or more determined surgical using a second trained neural network wherein the second trained neural network is trained to identify a representative image or video clip from the surgical video data based on the one or more determined surgical events; and outputting the report summary.

The methods and apparatuses (e.g., systems, software, etc.) described herein provide a technical solution to the technical problem of automatically and efficiently generating reports and/or guiding surgical procedures. These methods and apparatus may allow real-time (or near-real-time) automated analysis of even complex surgical procedures to generate text and/or video report summaries that include representative images or clips without requiring laborious and time-consuming manual review. These methods and apparatuses enhance the automatic or semi-automatic operation of the hardware (e.g., one or more processors) in extracting relevant information, allowing them to operate faster and more efficiently.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 shows an example system for processing and managing surgical media.

FIG. 2 is a flowchart showing an example method for managing and processing media, particularly surgical video data.

FIG. 3 shows a block diagram of a device 300 that may be one example of the AI media processor of FIG. 1.

### DETAILED DESCRIPTION

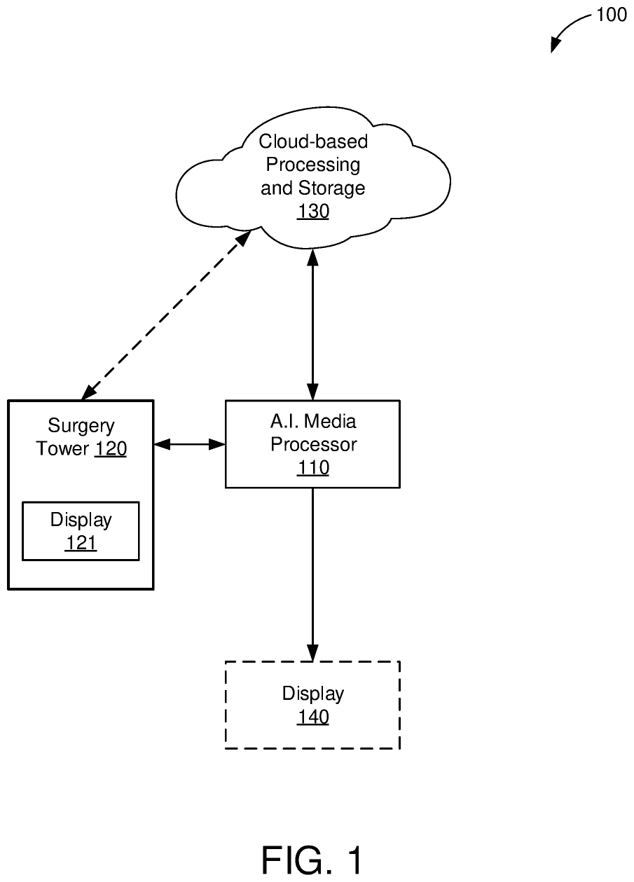

FIG. 1 shows an example system 100 for processing and managing surgical media. The system 100 may include an artificial intelligence (AI) media processor 110, a surgery tower 120, a cloud-based processing and storage unit 130, and an optional display 140. The system 100 may receive, manage, and store surgical media data, including surgical video data. Furthermore, the system 100 may analyze the surgical video data to provide a surgeon, doctor, or other clinician with three-dimensional (3D) surgical navigation, capture and store any related processed surgical video, and generate reports related to surgical operations.

The cloud-based processing and storage unit 130 may be any feasible remote storage and processing device, facility, or the like. In some variations, access to the cloud-based processing and storage unit 130 may be controlled and limited to authorized users and, in some cases, access may be encrypted. Thus, the cloud-based processing and storage unit 130 may provide secure, remotely accessible data storage. The cloud-based processing and storage unit 130 may include one or more processors, memory devices, and network interfaces. Thus, the cloud-based processing and storage unit 130 be able to execute programs, algorithms, and in some cases, trained neural networks. In some examples, the AI media processor 110 may be coupled directly or indirectly to the cloud-based processing and storage unit 130 through any feasible network, including the Internet.

In any of the methods and apparatuses described herein the processing may be divided or split between cloud and local computation. For example, it may be particularly beneficial to divide processing between local processor (e.g., for low-latency needs) and remote processor(s) (e.g., for high-latency needs). Some components may be processed both locally and remotely, while others may be separately handled. This architecture, in which local and remote processing is divided up, may be streamlined as described in greater detail herein as when handling low-bandwidth situations.

The cloud-based processing and storage unit 130 may receive and/or store standard (conventional) and surgeon-specific diagnostic procedural checklists regarding patient surgeries. In some variations, the cloud-based processing and storage unit 130 may receive and/or store surgery guidance modules for one or more surgical procedures. The cloud-based processing and storage unit 130 may also receive and/or store one or more generic models of anatomical joint that may be associated with any feasible surgery.

The cloud-based processing and storage unit 130 may receive and/or store patient-specific information. For example, the cloud-based processing and storage unit 130 may receive and/or store a patient's radiological imaging data as well as a patient-specific preoperative plan.

Any data stored within the cloud-based processing and storage unit 130 may be uploaded directly or indirectly by a doctor, surgeon, clinician or other medical professional. In some examples, the data stored within the cloud-based processing and storage unit 130 may be retrieved from one or more remote databases.

The AI media processor 110 may include one or more processors, a memory, input/output interface devices, and a graphical processing unit (GPU). The input/output interface devices may enable wired and/or wireless network communications, and enable the reception of video data (surgical video data) through HDMI ports, USB ports, or any other feasible connection to receive streaming video data. The processors, memory, and GPU may enable the AI media processor 110 to process and analyze surgical video data as well as execute one or more trained neural networks.

The AI media processor 110 may receive surgical video data either from the surgery tower 120 or the cloud-based processing and storage unit 130. In some variations, the surgery tower 120 may be coupled to endoscopic or arthroscopic surgery equipment and receive video data from an endoscopic or arthroscopic camera. In some variations, the AI media processor 110 may receive video data from the surgery tower through an HDMI port, a USB port, or any other feasible port or connector. The AI media processor 110 may then process the surgical video data with one or more trained neural network models to infer surgical information and/or guidance for the surgeon. In some examples, the AI media processor 110 may retrieve one or more neural network models that are stored in the cloud-based processing and storage unit 130. The AI media processor may generally retrieve models based on a determination of the surgical context.

In some variations, the AI media processor 110 can simultaneously receive surgical video data through a network connection and through a USB or HDMI port. Then, in a low-bandwidth situation, surgical video data may be uploaded to the cloud-based processing and storage unit 130 while a surgery is taking place and captured images and other patient information may be received after the surgical procedure. In this manner, the system 100 may use compute resources within the cloud-based processing and storage unit 130 to process the surgical video data and provide any results sooner to the user.

The AI media processor 110 may provide a 3D navigational guide to the surgeon. The 3D navigational guide may provide real-time surgical guidance to the surgeon regarding planned surgery for the patient. In some variations, the 3D navigational guide may include graphical overlay information that may be combined or superimposed over surgical video data. The AI media processor 110 may generate the 3D navigation guide by executing one or more trained neural network models to process the surgical video data, patient-specific information such as a patient's radiological imaging data as well as the patient's preoperative plan. In some examples, if the patient's radiological imaging data is not available, the AI media processor 110 may generate the 3D navigational guide based on one or more generic models of anatomical joints.

The 3D navigational guide may be displayed on a display 121 included within the surgery tower 120. In some variations, the AI media processor 110 may display the 3D navigational guide on a separate (optional) display 140.

In general, the methods and apparatuses described herein may provide an output. The For example, the output of the real-time analysis described herein can be the original video stream and one or more AI overlays, which may be sent to a standalone monitor or a suitable receptacle. In some examples, the output could also be just the overlay, in which case, the recipient may themselves blend with the original input stream and send, for example, to display (and/or storage for later display).

The AI media processor 110 may analyze the surgical video data, which may include a real-time surgery video stream, to provide real-time graphical assistance as well as record and log surgical data associated with a patient's surgery. The AI media processor 110 may execute one or more trained neural networks to infer data used to provide the graphical assistance and guide the recording and storage of the surgical data.

For example, the AI media processor 110 may execute a neural network trained to detect a type of surgery as well as detect a joint or anatomical region related to the location of the surgery from the surgical video data. The AI media processor may display a 3D navigational guide that highlights the position of an endoscopic or arthroscopic camera (sometimes referred to as an endoscope, arthroscope or scope). The AI media processor 110 may display the location of the endoscope or arthroscope, pinless landmarks, measurements and the like with respect to generic models of the anatomical joint or the patient's radiological imaging data. In some variations, the location of the endoscopic or arthroscope, pinless landmarks, measurements and the like may be displayed with more accuracy when using the patient's radiological imaging data.

The AI media processor 110 may execute a neural network trained to understand and/or recognize the surgical area. For example, the AI media processor 110 may process surgical video data and provide surgical safety warnings or outline areas of concern in and around the surgical area. In addition, the AI media processor 110 may determine whether any surgical procedures performed or anticipated will provide an optimal anatomical function. In some variations, the AI media processor 110 may execute a neural network trained to identify any feasible anatomical structure.

The AI media processor 110 may execute a neural network trained to understand and/or recognize various surgical aspects from the surgical video data. For example, the AI media processor 110 may process the surgical video data to understand and/or recognize any feasible surgery stage, surgical activity, and surgical action. In addition, the AI media processor 110 may process the surgical video data to recognize surgical tools, surgical implants, sutures, and/or anchors. The AI media processor 110 may generate a log file that notes or records any of the surgical aspects recognized and described herein. Thus, the AI media processor 110 may record the recognition of any surgery stage, surgical activity, surgical action, surgical tools, surgical implants, sutures, and/or anchors. In some variations, the AI media processor 110 may also recognize and record anatomical locations where implants were used. In addition or alternatively, the apparatuses and methods described herein may be configured to automatically select the best frame (image) which represents a stage or activity. This may be helpful for generating one or more reports (e.g., which may be used for, e.g., insurance billing purposes).

The AI media processor 110 may execute a neural network trained to recognize locations where the surgeon has established pinless/virtual landmarks within a surgical area. In some variations, the AI media processor 110 may execute a neural network trained to recognize any measurements performed by the surgeon. In some variations, the AI media processor 110 may store and/or record any surgical data associated with pinless/virtual landmarks and measurements. For example, the AI media processor 110 may store determined locations of various pinless/virtual landmarks and locations associated with measurements performed by the surgeon.

The AI media processor 110 may execute a neural network trained to recognize the presence or lack of surgical progress. For example, the AI media processor 110 may process surgical video data and determine whether an endoscopic or arthroscopic camera is withdrawn from the surgical area before surgical repairs are complete. In some cases, the neural network may be trained to detect and/or identify surgical procedures. The AI media processor 110 may process surgical video data and determine whether the endoscopic or arthroscopic camera is in or near an anatomical joint without recognizing any surgical procedures. In some variations, the AI media processor 110 may process surgical video data and determine whether the endoscopic or arthroscopic camera has been in or near an anatomical joint multiple times with a surgical tool, however the surgery stage has not advanced.

The AI media processor 110 may execute a neural network trained to recognize or detect novel scenes within the surgical video data. In some aspects, a novel scene may refer to surgical video data associated with a surgical procedure for which there is no trained neural network. Thus, the AI media processor 110 may detect scenes or surgical activities it is unable to recognize and classify with confidence. The AI media processor 110 may generate alerts to the user indicating that the system 100 has encountered scenes (surgical video data) for which it has not been trained.

In some examples, after the AI media processor 110 detects a novel scene, then the AI media processor 110 may deactivate visual overlays and/or guidance that may be provided to the user. For example, assistance data may no longer be displayed with any 3D navigation information. The AI media processor 110 may also isolate and record any associated surgical video data associated with the novel scene as well as any feasible video context. The surgical video data may be recorded and saved in any feasible location. In some examples, the surgical video data may be saved locally in the AI media processor 110. In some other examples, the surgical video data may be saved in the cloud-based processing and storage unit 130. In some variations, the surgical video data may be “deidentified.” Deidentification may include removing or redacting patient-specific data or metadata. The deidentified surgical video data may be encrypted and uploaded (stored) in any feasible location. In some cases, the deidentified surgical video data may be stored in the cloud-based processing and storage unit 130.

In some examples, the AI media processor 110 may execute a neural network trained to create summary data associated with a surgical procedure. The summary data may include any feasible documentation, including text-based descriptions of any feasible, identified, or recognized surgical procedures. The summary data may also include any feasible video data (e.g., video clips and/or still images) captured or derived from the surgical video data. In some variations, the summary data may be generated in real-time based on the surgical video data received from the surgery tower 120 or any directly or indirectly coupled endoscopic or arthroscopic camera, or the like. In some examples, execution of the neural network trained to create summary data may cause the AI media processor 110 to automatically select an appropriate scene to represent each surgery stage. The AI media processor 110 may automatically detect a beginning and an end of a scene that includes any distinct surgical action and generate an associated video clip. In some variations, the AI media processor 110 may automatically generate a text summary to accompany any video clip. As mentioned, any of the methods or apparatuses described herein may be configured to identify the general/generic beginning of the surgery (surgical activity) and to progressively and/or separately identify more specific surgical procedures. In any of these methods and apparatuses the technique may parse or divide the identified surgical portions into (linked) stages and may use this for surgical activity detection and/or analysis.

Any of the summary data may be uploaded or transferred to another file system. In some examples, any of the text-based descriptions may be converted into PDF files. Any of the summary data (files, still images, and or video data) may be transmitted to an electronic health record system through an electronic fax interface, an email interface, or a suitable HL7 interface that may be included with the system 100.

In some examples, the AI media processor 110 may execute a neural network trained to receive voice inputs from the surgeon during the time of the surgery. The AI media processor 110 may convert the voice inputs into text and match the text to any surgical activity. In some variations, the matching may be based on any machine recognition of patient anatomy, patient pathology, surgical tool, anatomical joint view, region, pinless landmarks, and/or performed measurements. In some cases, the voice inputs may be used to generate context-specific alerts which are then pushed or transferred to any appropriate system. Alternatively or additionally, voice inputs may be use as annotation/supplemental information (and not, strictly speaking, an alert). For example, a surgeon's voice annotations may be attached to the report at an appropriate juncture; for instance, when examining a joint, the surgeon might add an observation about the health of the cartilage. When the methods and apparatuses described herein generate a surgery report, this voice input may be attached/annotated to the appropriate stage along with a ‘most representative’ image corresponding to the stage. This function may help a surgeon to better recall when generating op-notes, submit insurance claims, and generate evidence to support medical necessity claims.

In some examples, the AI media processor 110 may generate a log of pinless landmarking and measurements made or used during a surgical procedure. In some other examples, the AI media processor 110 may generate alerts and/or notifications regarding surgical activity to an external database or through any feasible secure network connection. For example, the AI media processor 110 may store alerts and/or notifications within the cloud-based processing and storage unit 130 or any feasible network drive. Alternatively, or in addition, the AI media processor 110 may write the alerts and/or notifications to a suitable queuing system that processes such alerts. In some examples, the AI media processor 110 can save any summary data, logfile, or report to a secure backend database.

The AI media processor 110 may playback any feasible video data, including surgical video data. The video data may be displayed on the display 121 in the surgery tower 120 or the display 140. Using the playback data, the surgeon may modify or alter any output files, reports, or modify the training of any neural networks. In some examples, the surgeon may add a voice annotation associated with any surgical video data.

In some examples, the AI media processor 110 may store the surgical video data wholly or in parts. The surgical video data may be stored in a secure cloud storage (such as the cloud-based processing and storage unit 130).

In some cases, the surgical video data may be deidentified. In one example of deidentification, the AI media processor 110 may execute a neural network trained to detect when the endoscopic or arthroscopic camera is outside or beyond a particular anatomical joint. When the endoscopic or arthroscopic camera is outside or beyond a particular joint, the AI media processor 110 may delete associated surgical video data. In another example of deidentification, the AI media processor 110 may also remove any patient-specific information from the surgical video data including, but not limited to, surgery location, data, time, patient's name. In some examples, the AI media processor 110 may execute a neural network trained to identify and extract surgical actions into video clips. The video clips may have any patient-specific metadata removed.

In some examples, the AI media processor 110 may processes any feasible surgical video data to modify color balance or color palette. For example, the AI media processor 110 may modify colors to a predetermined color palette, such as customer-or application-specific color palette.

In general, the methods and apparatuses described herein may direct the storage of the video data (and/or additional data, including annotations, analysis, etc.) being performed to ensure storage fidelity even as the local bandwidth, processing and memory concerns may change. For example, if a network connection is unavailable, then the AI media processor 110 may store any surgical video data locally until access to remote storage is provided. If local storage space is limited, then the AI media processor 110 may overwrite any oldest recordings.

In some examples, the AI media processor 110 may store surgical video data locally (for example, in a memory within the AI media processor 110). The AI media processor 110 may receive one or more neural network models, in some cases from the cloud-based processing and storage unit 130. The AI media processor 110 may then execute the received neural network model on the surgical video data and upload the results back to the cloud-based processing and storage unit 130. In this manner, the surgical video data can remain within the clinician's environment while only results are uploaded to a secure cloud storage. In some variations, the received neural network models may also executed on real-time surgical video data.

In any of these examples, the local processor may analyze the stored content, extract various attributes (e.g., surgery type, region, tools, anatomical structures, stages covered, etc.) and may submit these attributes to the remote server. These attributes may be matched against those of the models which are candidates for remote inferencing. The matched model(s) may be sent to the AI media processor for analysis. The results may be sent back, associated with the specific models(s) sent and the findings evaluated.

The AI media processor 110 may operate in a training mode where overlays, guidance and results of the execution of any neural network models are provided to a clinician or surgeon. The clinician or surgeon may provide feedback and/or observations associated with AI media processor 110 operations. The feedback and/or observations may be uploaded to the cloud-based processing and storage unit 130 and used to refine or further train any of the neural network models.

In some variations, functionality of the AI media processor 110 may be included within the cloud-based processing and storage unit 130. Thus, any of the operations performed by the AI media processor 110 may be performed wholly or in part by the cloud-based processing and storage unit 130.

In some variations, any or all of the functionality of the system 100 may be combined or included with any other feasible device. Including the functionality of the system 100 within other devices may reduce implementation costs and advantageously share devices, such as displays, input/output devices and the like.

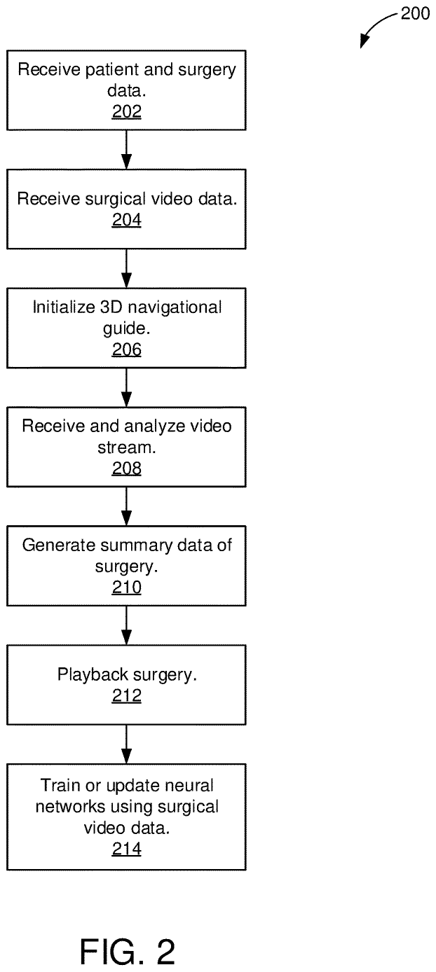

FIG. 2 is a flowchart showing an example method 200 for managing and processing media, particularly surgical video data. Some examples may perform the operations described herein with additional operations, fewer operations, operations in a different order, operations in parallel, and some operations differently. The method 200 is described below with respect to the system 100 of FIG. 1, however, the method 200 may be performed by any other suitable system or device.

The method 200 begins in block 202 as the system 100 receives patient and surgery data. For example, the AI media processor 110 may receive a patient-specific preoperative plan and/or the patient's radiological imaging data.

Next, in block 204 the system 100 may receive surgical video data. For example, the AI media processor 110 may receive a video stream from an endoscopic or arthroscopic camera, from the cloud-based processing and storage unit 130, from any feasible network, or the like. In some variations, the system 100 may store the surgical video data locally (in the AI media processor 110) or within the cloud-based processing and storage unit 130.

Next, in block 206 the system 100 initializes a 3D navigational guide. The 3D navigational guide may include display overlays that provide the real-time guidance regarding a planned surgery for the patient. In some variations, the 3D navigational guide may provide guidance information regarding the positioning of an endoscopic or arthroscopic camera or surgical tools within an anatomical joint. If the patient's radiological imaging data is not available, then the AI media processor 110 may generate the 3D navigational guide based on one or more generic models of any feasible anatomical joint.

Next, in block 208 the system 100 may analyze the surgical video data. For example, the AI media processor 110 may execute any of the one or more trained neural networks described herein to process the received video stream of the surgical video data. The AI media processor 110 may update the 3D navigational guide with a current position of the endoscope, arthroscope, or other camera. The AI media processor 110 can review and recognize the surgical area in real-time to understand surgery stages, surgical activity, surgical actions, and the like. The AI media processor 110 may also recognize and log the use of implants, sutures, anchors, pinless and virtual landmarks. In some variations, the AI media processor 110 can recognize, track, and log surgical progress. The AI media processor 110 may also detect novel scenes during the patient's surgery.

Next, in block 210 the system 100 generates summary data of the patient's surgery. For example, the AI media processor 110 may generate a summary video that includes textual highlights of the surgical procedure. The AI media processor 110 may execute a neural network to automatically select an appropriate scene for every surgery stage. The summary data may include information regarding the type of implant used, the number of anchors used (if any), and may include voice notes from the surgeon. The summary data may be stored locally and/or in the cloud-based processing and storage unit 130.

Next, in block 212 the system 100 plays back the surgical video data. In some cases, the AI media processor 110 may playback highlights of the surgery. In some examples, the surgeon can add additional voice narration to the surgical video data.

Next, in block 214, the system 100 may train or update one or more neural network models using the surgical video data. For example, the AI media processor 110 may train neural networks using the surgical video data received in block 204, or surgical video data received from the cloud-based processing and storage unit 130. In some variations, the training may be guided by inputs from the surgeon.

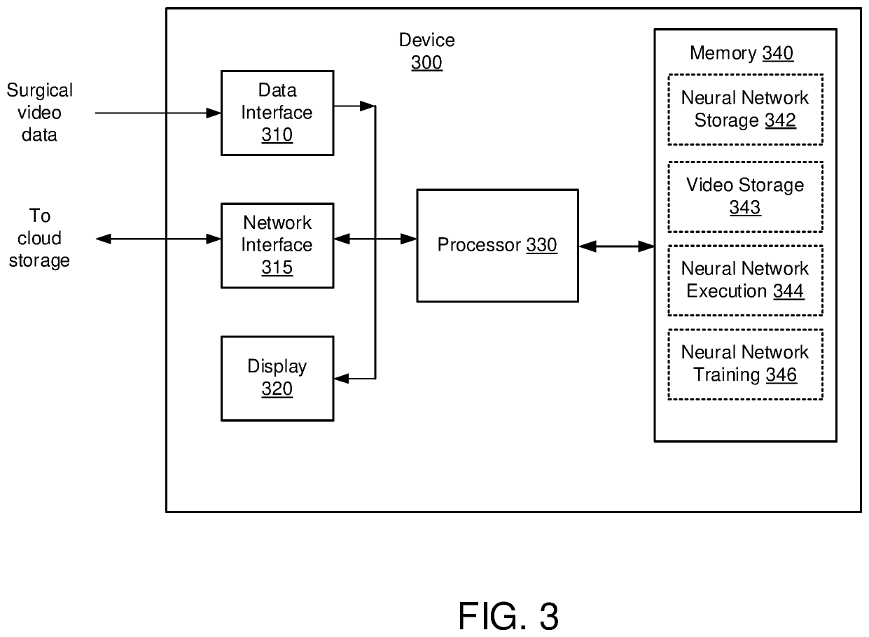

FIG. 3 shows a block diagram of a device 300 that may be one example of the AI media processor 110 of FIG. 1. The device 300 may include a data interface 310, a network interface 315, a display 320, a processor 330, and a memory 340.

The data interface 310, which is coupled to the processor 330, may be used to receive video data, including real-time surgical video data. In some examples, the data interface may include HDMI ports, USB ports, or any other feasible port to receive video data. In some implementations, the data interface 310 may be coupled to a camera such as an endoscopic or arthroscopic camera.

The network interface 315, which is also coupled to the processor 330, may be used to transfer data with any other feasible device or network. For example, the network interface 315 may be coupled to the cloud-based processing and storage unit 130. In some variations, the network interface 315 may include any feasible communications devices or circuits including, but not limited to, wired and wireless communication circuits. In particular, any of the methods and apparatuses described herein may be configured to be used with one or more network-based shared drives.

The display 320, which is also coupled to the processor 330, may be used to display or playback video data, including surgical video data. The display 320 may also be used to display 3D navigational guidance to assist the surgeon during any feasible surgery.

The processor 330, which is also coupled to the memory 340, may include any one or more suitable processors capable of executing scripts or instructions of one or more software programs stored in the device 300 (such as within memory 340).

The memory 340 may include a neural network storage 342 that may be used to locally store one or more trained neural networks (e.g., neural network models). In some cases, neural network models may be received through the network interface 315 and then stored within the neural network model storage 342.

The memory 340 may include a video storage 343 that may be used to locally store video data, including surgical video data received through the data interface 310 and/or the network interface 315.

The memory 340 may also include a non-transitory computer-readable storage medium (e.g., one or more nonvolatile memory elements, such as EPROM, EEPROM, Flash memory, a hard drive, etc.) that may store the following software modules: a neural network execution module 344 to execute one or more neural networks; and a neural network training module 346 to train one or more neural networks.

Each module may include program instructions that, when executed by the processor 330, may cause the device 300 to perform the corresponding function(s). Thus, the non-transitory computer-readable storage medium of memory 340 may include instructions for performing all or a portion of the operations described herein.

The processor 330 may execute the neural network execution module (“neural network model”) 344 to execute and perform any feasible neural network described herein. The neural network models may be stored in the neural network storage 342. Execution of the neural network models may enable the processor 330 to analyze video data for surgical content including anatomies, implants, sutures, and the like. Execution of the neural network models may also enable the processor 330 to generate summary data including a summary video based on surgical video data.

The processor 330 may neural network training module 346 to train any feasible neural network. In some variations, the processor 330 may train any feasible neural network using video data, in some cases video data stored in the video storage 343. In some examples, execution of the neural network training module 346 may enable the device 300 to receive inputs from a surgeon to help train any feasible neural network.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A method for managing surgical data, the method comprising:
receiving surgical video data;
applying a trained first neural network to the surgical video data to determine one or more surgical events based on the surgical video data, wherein the is first trained neural network is trained to recognize at least one of: a surgery stage, a surgical activity, a surgical action, a surgical tool, an implant, a suture, and an implant anchor; and
forming a report summary including a video clip comprising highlights of one or more determined surgical using a second trained neural network wherein the second trained neural network is trained to identify a representative image or video clip from the surgical video data based on the one or more determined surgical events; and
outputting the report summary.

2. The method of claim 1, wherein the second neural network is trained to indicate that no surgical progress was made during a related surgery in the video clip.

3. The method of claim 1, further comprising:
receiving patient-specific radiological imaging information; and
generating a three-dimensional (3D) navigational guidance for a surgery based, at least in part, on the patient-specific radiological imaging information.

4. The method of claim 3, wherein the 3D navigational guidance is based at least in part on a patient-specific preoperative plan.

5. The method of claim 3, wherein the 3D navigational guidance is based at least in part on generic models of an anatomical joint.

6. The method of claim 1, further comprising generating a deidentified video clip based at least in part on the surgical video data.

7. The method of claim 1, wherein the report summary includes a text summary of one or more surgical stages within the surgical video data.

8. The method of claim 1, wherein forming the report summary comprises generating a log of pinless landmarks used and measurements taken during a surgery associated with the surgical video data.

9. The method of claim 1, wherein outputting comprising saving the report summary to a secure cloud-based storage unit.

10. The method of claim 1, wherein the surgical video data is a real-time video stream received from an endoscopic camera.

11. The method of claim 1, wherein the surgical video data is received from a secure cloud-based storage unit.

12. The method of claim 1, wherein the surgical video data is received via an HDMI or USB connector.

13. The method of claim 1, further comprising generating an alert based on a recognized surgical activity.

14. The method of claim 1, further comprising:
receiving a voice input from a surgeon during the surgery; and
generating context specific alerts based on the voice input.

15. The method of claim 1, wherein the first neural network is trained to determine a start and stop of a surgical action.

16. A system, comprising:
one or more processors; and
a memory configured to store instructions that, when executed by one of the one or more processors, cause the system to:
apply a trained first neural network to a surgical video data to determine one or more surgical events based on the surgical video data, wherein the is first trained neural network is trained to recognize at least one of: a surgery stage, a surgical activity, a surgical action, a surgical tool, an implant, a suture, and an implant anchor; and
form a report summary including a video clip comprising highlights of one or more determined surgical using a second trained neural network wherein the second trained neural network is trained to identify a representative image or video clip from the surgical video data based on the one or more determined surgical events; and
output the report summary.

17. A non-transitory computer-readable storage medium comprising instructions that, when executed by one or more processors of a device, cause the one or more processors to perform operations comprising:
applying a trained first neural network to a surgical video data to determine one or more surgical events based on the surgical video data, wherein the is first trained neural network is trained to recognize at least one of: a surgery stage, a surgical activity, a surgical action, a surgical tool, an implant, a suture, and an implant anchor; and
forming a report summary including a video clip comprising highlights of one or more determined surgical using a second trained neural network wherein the second trained neural network is trained to identify a representative image or video clip from the surgical video data based on the one or more determined surgical events; and
outputting the report summary.

18. The non-transitory computer-readable storage medium of claim 17, wherein the second neural network is trained to indicate that no surgical progress was made during a related surgery in the video clip.

19. The non-transitory computer-readable storage medium of claim 17, wherein the instruction are further configured to cause the one or more processors to perform the operations comprising:
receiving patient-specific radiological imaging information; and
generating a three-dimensional (3D) navigational guidance for a surgery based, at least in part, on the patient-specific radiological imaging information.

20.-29. (canceled)

30. The non-transitory computer-readable storage medium of claim 17, wherein the instruction are further configured to cause the one or more processors to perform the operations comprising:
receiving a voice input from a surgeon during the surgery; and
generating context specific alerts based on the voice input.

31. (canceled)
